(19) United States
(12) Patent Application Publication
(10) Pub. No.: US 2003/0228321 A1
(43) Pub. Date: Dec. 11, 2003

(54) Preventives for reinfection after liver transplantation

(76) Inventors: Takafumi Ichida, Niigata (JP); Yoshiharu Kawashima, Yokohama (JP)

Correspondence Address: SCHNADER HARRISON SEGAL & LEWIS, LLP, 1600 MARKET STREET, SUITE 3600, PHILADELPHIA, PA, 19103

(21) Appl. No.: 10/258,206

(22) PCT Filed: Apr. 20, 2001
(86) PCT No.: PCT/JP01/03385

(30) Foreign Application Priority Data
Apr. 20, 2000 (JP) 2000-119174

(51) Int. Cl. A61K 39/00; A61K 39/38

(52) U.S. Cl. 424/184.1

(57) ABSTRACT

Reinfection with the hepatitis C virus after liver transplantation is protected by administering IFN-β before liver transplantation, and thus IFN-β is useful as a reinfection protecting agent.

### TECHNICAL FIELD

The present invention relates to a reinfection protecting agent comprising interferon-β as an effective ingredient.

### BACKGROUND ART

A liver disease with the hepatitis C virus (HCV) is a disease which progresses stepwisely from chronic hepatitis due to persistent infection with. HCV to cirrhosis, and further progresses to hepatocellular carcinoma (HCC), and liver transplantation is applied to cirrhosis and HCC. However, HCV is known to cause substantially 100% reinfection of a grafted liver after transplantation. Therefore, at present, as a protective measure against reinfection, the amount of the immunosuppressive agent used after transplantation is decreased, or combined therapy using interferon-α and ribavirin is used when a postoperative liver disorder is recognized.

On the other hand, an angiogenetic inhibitory action, a (bFGF) production inhibitory action, and a tumor cell wetting inhibitory action are observed in interferon (referred to as “IFN” hereinafter) (Sidky Y A, et al.: Inhibition of Angiogenesis by Interferon: Effect on Tumor- and Lymphocyte-induced Vascular Response. Cancer Res. 47, 5155-5161 (1987)), (Gohji K., et al., Human Recombinant Interferons-Beta and Gamma Decrease Gelatinase Production and Invasion by Human KG-2 Renalcarcinoma Cells. Int. J. Cancer 58, 380-384 (1994)). Also, IFN is known to have a therapeutic effect on the hepatitis C virus (Hiroshi Suzuki, et al.: Treatment of Chronic Active Hepatitis C with Human Interferon-β—a multicenter, randomized, controlled, trial with different duration of administration corresponding to viral load-, KAN•TAN•SUI 38, 571-589, 1999), but a protective effect on reinfection after liver transplantation is not known.

As the protective measure against reinfection after liver transplantation, the above-described method of decreasing the amount of the immunosuppressive agent has an insufficient effect, and reinfection with HCV occurs after liver transplantation, and progresses to chronic hepatitis and cirrhosis, or in some cases, hepatitis progresses to fulminant hepatitis within several years after operation. The combined therapy using IFN-α and ribavirin after operation exhibits an insufficient effective ratio, and there is thus demand for a therapeutic method for sufficient protection against reinfection.

### DISCLOSURE OF INVENTION

The present invention provides a protective agent against reinfection after liver transplantation, which comprises IFN-β as an effective ingredient. Furthermore, the present invention provides a protective method against reinfection after liver transplantation, comprising administering an agent comprising IFN-β as an effective ingredient.

### BEST MODE FOR CARRYING OUT THE INVENTION

The present invention relates to a protective agent against reinfection after liver transplantation, comprising IFN-β as an effective ingredient. The IFN-β used in the present invention may be a natural type, a type produced by chemical synthesis, or a type produced by a gene recombination technique. In the present invention, natural IFN-β is preferably used, and natural IFN-β produced by human fibroblasts is more preferably used.

The natural IFN-β can be produced by a method characterized in that a tumorigenic strain and virus are not used. The IFN-β produced in a culture solution is generally a low concentration, and the solution contains many foreign matters derived from cells or additives other than IFN-β. Therefore, the solution is concentrated and purified by a chromatography method using a blue pigment-bound insoluble carrier and a metal chelate group-bound carrier. However, the concentration and purification method is not limited to this.

In the present invention, IFN-β was administered to a patient of HCC complicated with HCV-related cirrhosis before liver transplantation from a living donor As a result, HCV in the blood continuously disappeared, and reinfection was not recognized. Therefore, the reinfection protecting effect of IFN-β after liver transplantation was confirmed. Based on this result, the protective agent against reinfection can be applied to patients of cirrhosis and HCC caused by HCV in order to conduct liver transplantation such as liver transplantation from a living donor, liver transplantation from a brain-dead donor, or the like. Although IFN-β may be administered before or after liver transplantation, particularly, the agent of the present invention is preferably administered before liver transplantation. Furthermore, the present invention can possibly be applied to the prevention of progress to chronic hepatitis and cirrhosis after operation, or progress to fulminant hepatitis within several years from operation.

Furthermore, a stabilizer can be added to the protective agent against reinfection of the present invention according to demand. Examples of the stabilizer include human serum albumin, the polyols disclosed in Japanese Unexamined Patent Application Publication No. 58-92619, the organic acid buffers disclosed in Japanese Unexamined Patent Application Publication No. 58-92621, and the like. Furthermore, a carrier in common use may be appropriately mixed with the agent to form a drug product according to the administration method used. Although an injection is used as a dosage form, the dosage form is not limited to this, and various forms such as a capsule agent, a nasal agent, a suppository, a oral agent, an ointment, and the like may be used. The present invention includes use of IFN-β for the manufacture of a protective agent against reinfection after liver transplantation.

A reinfection protecting method of the present invention has the requirement that an agent comprising IFN-β produced as described above as an active component is administered to a patient.

In clinical use of the reinfection protecting agent of the present invention, the dosage is appropriately determined according to the object of administration, the administration method, symptoms, etc., but the dosage is preferably in the range of 3,000,000 to 6,000,000 units per day.

In the present invention, the term “HCC” is used because 95% or more of liver cancer in Japan is HCC derived from hepatic cells. As shown by The Japan Society of Hepatology, liver cancer generally represents HCC.

### EXAMPLE

The present invention will be described in further detail below with reference to examples.

A 62-year-old female patient of HCC complicated with HCV-related cirrhosis was intravenously administered with 6,000,000 units-of IFN-β for 11 days, and then underwent liver transplantation from a living donor. After transplantation, the amount of the virus in the blood was measured by HCV-RNA quantitative analysis (Amplicore method). HCV in the blood continuously disappeared after transplantation, and reinfection was not recognized (Table 1).

TABLE 1

| | Before | | During | | The day | Liver | | | | |
|---|---|---|---|---|---|---|---|---|---|---|
| | administration | | administration | | after | transplantation | After liver transplantation | | | |
| | 5 | 3 | First | Fifth | adminis- | (1 week after | 1 | 2 | 3 | 4 |
| | months | months | day | day | tration | administration) | month | months | months | months |
| HCV-RNA bDNA probe | 1.4 | 0.77 | | | | | | | | |
| method (Meq/ml)* | | | | | | | | | | |
| Amplicore method | | | negative | negative | negative | | negative | negative | negative | negative |
| (copies/ml)** | | | | | | | | | | |

*1 Meq/ml 10⁵ copies/ml (Amplicore method)
**<10² copies/ml: Negative

Industrial Applicability

The protective effect of IFN-β on virus reinfection after liver transplantation is suggested. This indicates that IFN-β is useful as a reinfection protecting agent.

1. A protective agent against reinfection after liver transplantation, comprising interferon-β as an effective ingredient.

2. A protective agent against reinfection after liver transplantation according to claim 1, wherein the liver transplantation is conducted for hepatitis C virus-related cirrhosis or hepatitis C virus-related hepatocellular carcinoma.

3. A protective agent against reinfection after liver transplantation according to claim 1, wherein the liver transplantation is liver transplantation from a living donor or liver transplantation from a brain-dead donor.

4. A protective method against reinfection after liver transplantation, comprising administering an agent comprising interferon-β as an effective ingredient.

5. Use of interferon-β for the manufacture of a protective agent against reinfection after liver transplantation.
